(19) United States
(12) Patent Application Publication
(10) Pub. No.: US 2007/0110756 A1
(43) Pub. Date: May 17, 2007

(54) Method for modulating P53 activity via methylation

(76) Inventor: Danny F. Reinberg, Warren, NJ (US)

Correspondence Address: Jane Massey Licata;Licata & Tyrrell P.C., 66 E. Main Street, Marlton, NJ, 08053, US

(21) Appl. No.: 11/559,524

(22) Filed: Nov. 14, 2006

Related U.S. Application Data
(60) Provisional application No. 60/736,253, filed on Nov. 14, 2005.

(51) Int. Cl. A61K 39/395; A61K 38/17

(52) U.S. Cl. 424/155.1; 514/12

(57) ABSTRACT

The present invention relates to the modulation of p53 activity based upon the stabilization of p53 via methylation by Set9. Stabilization or destablization of p53 is useful in methods for modulating apoptosis and treating cancer, either alone or in combination with exogenous p53 or antineoplastic agents. Antibodies which specifically recognize methylated p53 are also provided.

This application claims benefit of U.S. Provisional Patent Application Ser. No. 60/736,253, filed Nov. 14, 2005, the content of which is incorporated herein by reference in its entirety.

### BACKGROUND OF THE INVENTION

The transcription factor p53 is mutated in approximately 50% of human cancers. In normal cells, p53 exerts a pivotal role in controlling the cell cycle, apoptosis and DNA repair in response to various forms of genotoxic stress. The regulation of p53 is complex and occurs mainly at the post-translational level (Appella & Anderson (2001) Eur. J. Biochem. 268:2764-2772). This complexity is realized through the number and types of different post-translational modifications that contribute to its stabilization and activation. Phosphorylation of several residues at the amino terminus of p53 has been shown to affect its interaction with MDM2, which targets p53 for ubiquitin-mediated degradation (Shieh, et al. (1997) Cell 91:325-334; Momand, et al. (2000) Gene 242:15-29; Brooks & Gu (2003) Curr. Opin. Cell Biol. 15:164-171). The carboxyl terminus of p53 is rich in lysines, which are subjected to acetylation, ubiquitination and sumoylation (Appella & Anderson (2001) supra). Acetylation of the carboxyl terminus has been shown to protect p53 from ubiquitination (Li, et al. (2002) J. Biol. Chem. 277:50607-50611). Moreover, acetylation of p53 at lysines 373 and 382 increases its DNA-binding activity (Luo, et al. (2004) Proc. Natl. Acad. Sci. USA 101:2259-2264; Gu & Roeder (1997) Cell 90:595-606) and potentiates its interaction with other transcription factors (Barlev, et al. (2001) Mol. Cell. 8:1243-1254). The positive effects of acetylation on p53 activity can be reversed by deacetylation (Gu, et al. (2004) Nov. Found. Symp. 259:197-205). p53 has also been shown to be sumoylated at K386, although the exact role of this modification in the regulation of p53 is not yet clear (Gostissa, et al. (1999) EMBO J. 18:6462-6471; Kwek, et al. (2001) Oncogene 20:2587-2599; Rodriguez, et al. (1999) EMBO J. 18:6455-6461).

Various histone modifications such as phosphorylation, acetylation, methylation and ubiquitination have been implicated in the regulation of gene expression by several mechanisms. The interplay between these modifications has been studied in detail and a ‘histone code’ hypothesis has been proposed to explain the effects of these modifications on gene expression (Jenuwein & Allis (2001) Science 293:1074-1080). Recently, a histone methyltransferase, Set9, that targets histone H3 at lysine 4 was identified and it was shown that this modification is associated with gene activation (Wang, et al. (2001) Mol. Cell. 8:1207-1217; Nishioka, et al. (2002) Genes Dev. 16:479-489). Other substrates for this enzyme have not been extensively analyzed.

### SUMMARY OF THE INVENTION

The present invention is a method for modulating p53 activity by contacting p53 with Set9.

The present invention is also a method for modulating apoptosis in a cell by contacting the cell with an effective amount of Set9 or a Set9 inhibitor so that the p53 is respectively stabilized or destabilized. In one embodiment, the cell expresses wild-type p53. In other embodiments, the cell expresses a defective p53 and is contacted with exogenous wild-type p53. In a further embodiment, the cell is contacted with an antineoplastic agent.

The present invention is also a method for treating cancer by administering to a subject in need of treatment an effective amount of Set9 to stabilize p53. In one embodiment, cells of the cancer express wild-type p53. In other embodiments, cells of the cancer express a defective p53 and the subject is administered exogenous wild-type p53. In a further embodiment, the subject also receives an antineoplastic agent.

An antibody which specifically recognizes methylated p53 is also provided.

### BRIEF DESCRIPTION OF THE DRAWINGS

FIG. 1 is a schematic representation of the p53 domain structure and post-translational modification at the C-terminus. Circles, flags and the rectangle represent phosphorylation, acetylation and methylation of p53, respectively.

FIG. 2 shows the interaction of Set9 with p53 and H3 peptides. FIG. 2A is an alignment of amino acid residues adjacent to lysines of histone H3, p53, TAF10, and histone H4 targeted by methylation. Methylated lysine is underlined and the asterisk represents the consensus for substrate recognition by Set9 methyltransferase. FIG. 2B shows the dissociation constants of the p53 and H3 peptides as determined using a fluorometric competition assay. The unlabeled p53 and H3 peptides were used to displace a dansyl-labeled H3 10-mer of known affinity. The displacement curves were used to calculate the dissociation constants of the unlabeled peptides (shown in inset).

FIG. 3 shows that Set9 potentiates p53 function. U2OS cells were stably transfected with FLAG-Set9 wild-type, FLAG-Set9 mutant or siRNA against Set9. Non-transfected cells served as control. Cells were mock-treated, or treated with 0.3 μM of 5-fluoro-uracil (5-FU) or adriamycine (Adr) as indicated. p21 (FIG. 3A), BAX (FIG. 3B) and MDM2 (FIG. 3C) gene expression was analyzed by quantitative real-time PCR using primers specific for the coding region. Shown are the relative values normalized to GAPDH signal.

FIG. 4 shows that Set9-mediated activation of p21 gene expression requires p53. H1299 cells were stably transfected with FLAG-Set9 wild-type or control vector.

FIG. 5 shows methylation of p53 at the p21 promoter. U2OS and USOS Set9 siRNA cells were treated with 0.5 μM of Adr and collected at the indicated time points for a CHIP assay performed using anti-p53-Lys372me antibody. The amounts of precipitated p21 promoter were determined by quantitative RT-PCR.

### DETAILED DESCRIPTION OF THE INVENTION

It has now been found that Set9 methylates other proteins besides the histone polypeptides. In addition to several polypeptides, Set9 was found to methylate the tumor suppressor protein p53 in vitro. Under the assay conditions disclosed herein Set9-mediated methylation of p53 appeared to be specific. Other polypeptides such as cytochrome c, Ga14-VP16, bovine serum albumin (BSA) and γ-globulin did not serve as Set9 substrates. Moreover, other protein methyltransferases, such as Suv39H1, which targets H3-K9, PR-Set7 that targets H4-K20, or the arginine methyltransferase PRMT1, failed to use p53 as a substrate. Recent studies have indicated that Set9 can methylate TAF10 (Kouskouti, et al. (2004) Mol. Cell. 14:175-182), and although the studies herein did not address TAF10, the results collectively indicate that Set9-mediated methylation of proteins other than histone may be a general regulatory mechanism.

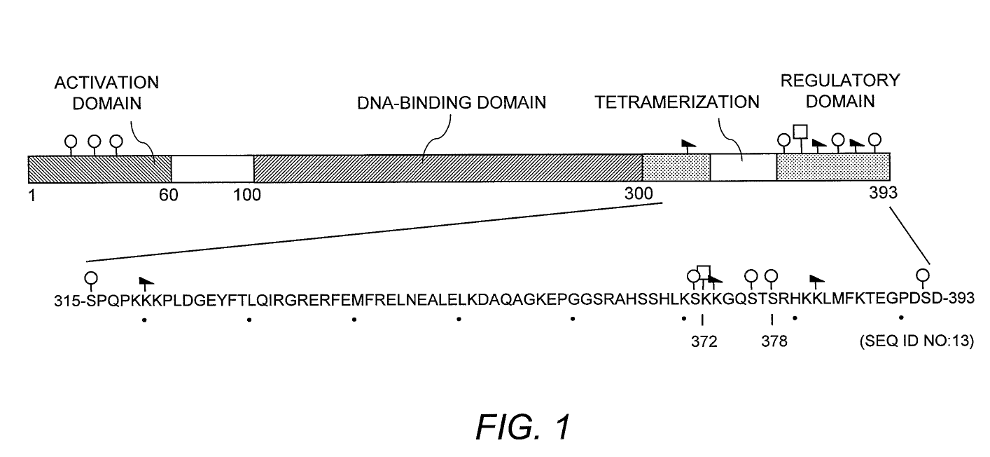

To gain insight into the role of p53 methylation, the p53 residue methylated by Set9 was mapped. The initial analysis was performed using three fragments of p53 encompassing its functional domains, the N-terminus (residues 1-82) representing the transactivation domain; the middle of the protein containing the DNA-binding domain (residues 96-312); and the regulatory C-terminus of the protein (residues 290-393). Set9 methylated p53 within the regulatory C-terminus. Subsequently, the C-terminus was further divided into three protein fragments, residues 289-325 containing 6 lysines, residues 340-364 containing 2 lysines, and residues 364-393 containing 6 lysines, and each fragment was analyzed for methylation by Set9. The most C-terminal fragment, extending over amino acids 364 to 393, was a substrate for methylation. As this peptide contained six lysines, each lysine was substituted individually, or in combination, with non-methylatable arginines and tested as substrates for methylation. The results of this analysis indicated that Set9 methylated lysine 372. Consistent with this observation, a single substitution at lysine 372 with arginine in the full-length p53 protein eliminated methylation by Set9. These data established that the site of Set9 methylation is within a region of the p53 protein that is subjected to multiple post-translational modifications (FIG. 1). This arrangement of amino acids, subjected to different post-translation modifications, resembles the arrangement within the histone tails, wherein one modification can affect another, an observation that has contributed in part to the histone code hypothesis.

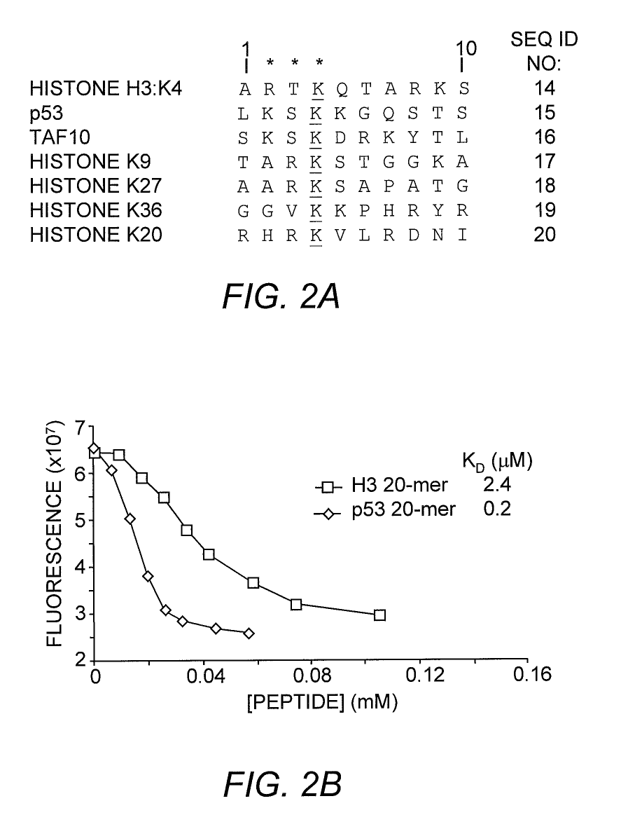

A comparison between the H3-K4 and p53 sequences shows amino-acid conservation around the methylation site on p53 at K372; H3 at K9, K27, K36; and H4 at K20. Moreover, if the amino-acid sequence present in TAF10, a substrate of Set9 (Kouskouti, et al. (2004) supra), is included in the analysis, the Set9 local recognition sequence appears to be restricted to three residues, Arg-Lys/Ser-Thr/Lys (FIG. 2A). The affinity of Set9 for putative peptide substrates derived from the sequences of either the H3 tail or p53 was determined by competition binding studies against an N-terminal dansyl-labeled H3 10-mer peptide (FIG. 2B). Notably, the affinity of the p53 20-mer was 12-fold higher than the equivalent H3 20-mer (a dissociation constant (K_{d}) of 0.2 μM compared to 2.4 μM, respectively). The tighter affinity for the p53 20-mer substrate correlated with a six-fold increase in enzymatic activity, as measured by methyltransferase assays with ³H-labeled S-adenosyl-L-methionine (AdoMet). As with a H3 peptide (Xiao, et al. (2003) Nature 421:652-656), Set9 mono-methylated the target lysine residue of p53.

The structure of Set9 in complex with a mono-methylated p53 peptide and the cofactor product AdoHcy was subsequently determined. This structure was compared to the previously solved complex of Set9 with mono-methylated H3 peptide (Xiao, et al. (2003) Nature). Well-ordered crystals of the p53 peptide ternary complex were obtained that diffracted to better than 1.8-Å resolution.

The overall structure of the complex was found to be similar to that obtained for the Set9-H3 ternary complex (Protein Data Bank (PDB) code lo9s). An overlay of Set9 ternary structures with p53 or H3 substrates showed a strong overall similarity between the two. Further, the mono-methylated lysine side-chain of the p53 peptide accesses the methyl donor cofactor using the same narrow channel running through the SET domain as that described for the H3 complex. Overall, the electron-density map for the Set9-p53 complex was of high quality. There was well-resolved electron density for the first six residues of the p53 peptide, for residues Leu (−3) to Gly (+2) (numbering is with respect to the modified p53 lysine residue 372) but the last four residues of the peptide were disordered. This observation indicates that Set9 interacts with a rather short recognition sequence at the active site of the enzyme. The Set9 residues involved in interactions with the three amino acids that are N-terminal to the modified lysine are the same for the p53 peptide and the H3 peptide, despite the difference in peptide sequence. The hydrogen bond between the carbonyl of Arg258 and the Nε atom of Arg (−2) in the H3 complex is replaced by a hydrogen bond with the Nε of Lys (−2) in p53. The hydrophobic packing of Trp260 with the peptide Arg (−2) in H3 is substituted with the packing against Lys (−2) in p53. There is also a conservative substitution of Ser (−1) in H3 for Thr (−1) in p53 that maintains the hydrogen bond with Ser268 of the enzyme.

Of the five Set9 residues making polar contacts with the peptide, four of them, Asp256, Arg258, Thr266 and Ser268, are located within the variable Set-I region. The sixth residue making a polar contact with the peptide is located in the C-flanking domain (Tyr335). Together, these observations highlight the significance of the variable Set-I and C-flanking domains in determining the specificity of the SET enzymes with respect to which lysine residue within a stretch of polybasic residues will be modified. The binding studies reported before also indicated that there must be amino-acid residues located at some distance from the target lysine residue that play an important role in determining substrate specificity. It is believed that the N-flanking domain, preceding the SET domain, may be involved in mediating these interactions.

To facilitate studies on the role of Set9-methylation of p53 in vivo, a polyclonal antibody was generated that specifically recognized mono-methylated p53-Lys372. Rabbit antibodies were initially screened against p53 peptides harboring Lys372 with different degrees of methylation. An antibody that specifically recognized a mono-methylated peptide, but failed to detect an unmodified peptide or equivalent peptides that were di-or tri-methylated was selected. The anti-p53-Lys372-mono-methyl (anti-p53-Lys372me) antibody specifically detected recombinant, purified p53 protein methylated by Set9 in vitro, but had less affinity for untreated p53 protein. As such, it was concluded that the antibody specifically recognized Lys372-methylated p53.

To determine whether p53 is methylated by Set9 in vivo, wild-type Set9 or its catalytically inactive mutant form (His297Ala) were stably transfected into 293F cells expressing endogenous p53. Western-blot analysis using anti-p53-Lys372me antibody detected methylated p53 protein in extracts derived from cells overexpressing the wild-type Set9 protein, but cells overexpressing the catalytically inactive Set9 protein displayed little methylated p53. As the expression levels of Set9 proteins and the amounts of p53 protein in the extracts were similar, it was concluded that transfected Set9 methylated p53 in 293F cells.

The in vivo methylation of p53 at lysine 372 under conditions in which the intracellular concentration of Set9 was unaltered was also determined. 293F cells were treated with adriamycine (Adr), an agent that induces DNA damage resulting in the activation of a p53-responsive pathway. Cell extracts prepared from untreated and Adr-treated cells were then compared for total amounts of p53 protein using monoclonal p53 antibody. Under these conditions, DNA damage did not increase the levels of intracellular p53. Both extracts were also subjected to immunoprecipitation with anti-p53-Lys372me antibodies followed by western blot analysis with p53-specific antibody. Adr treatment increased the intracellular concentration of methylated Lys372-p53 protein. Furthermore, the levels of Set9 remained unchanged after Adr treatment. p53 also became methylated in response to DNA damage in U2OS cells, wherein the introduction of Set9 short interfering RNA (siRNA) in these cells decreased Lys372 methylation of p53 upon Adr treatment which correlated with the decreased levels of Set9. These findings collectively indicated that methylation of p53 in response to DNA damage is a general phenomenon, rather than a cell-type-specific event.

To characterize the function of p53-Lys372 methylation, 293F cells were stably transfected with a Set9 expression vector. Extracts from these cells were then fractionated into nuclear and cytosolic fractions to determine the distribution of methylated p53-Lys372. These studies indicated that all the methylated p53-Lys372 was localized to the nucleus, although p53 was equally distributed between the nuclear and cytosolic fractions. Moreover, untransfected 293F cells also displayed methylated p53 in the nuclear fraction, ruling out the possibility that the nuclear localization of methylated p53-Lys372 was a consequence of Set9 overexpression.

It was subsequently determined whether Set9-mediated methylation of p53 affected its transcriptional activity. Focus was placed on one of the best-characterized p53 transcriptional targets, the p21/WAF/CIP gene, whose product is critical for cell-cycle arrest in the G1 phase. The effect of Set9-mediated p53-Lys372 methylation on transcription of the endogenous p21 gene was measured in U2OS cells using reverse-transcription polymerase chain reaction (RT-PCR). Overexpression of Set9 resulted in increased expression of p21, which correlated with increased levels of Lys372-methylated p53. Even in the absence of DNA damage, the levels of p21 expression in cells overproducing Set9 were higher than those in the parental non-transfected cells treated with the DNA-damaging agent. Further, overexpression of Set9 followed by Adr treatment resulted in a further increase in the levels of p21 expression.

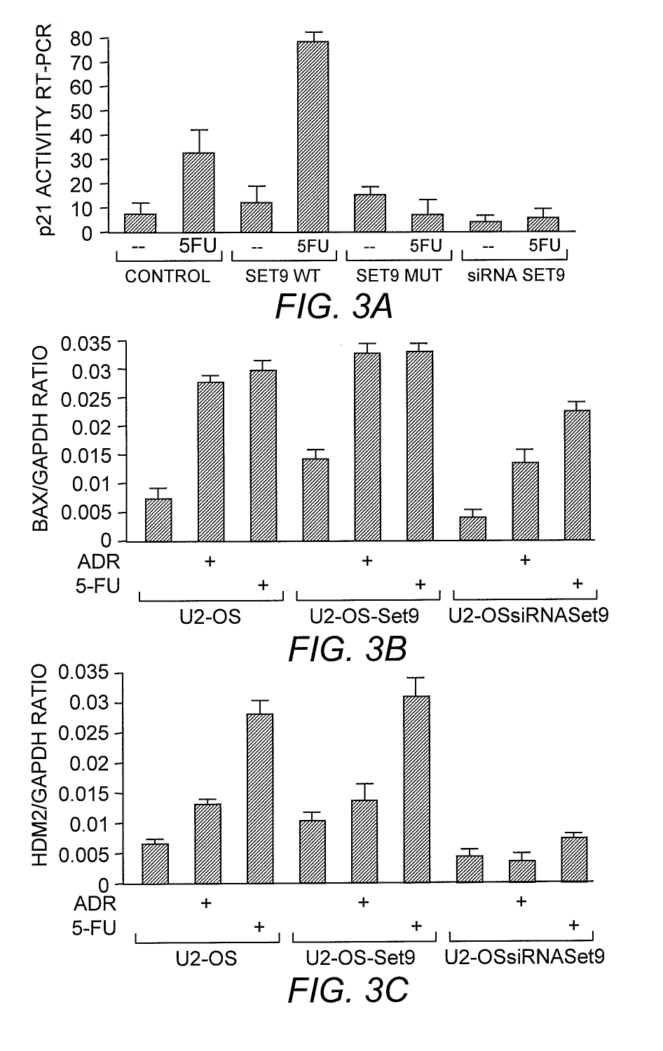

The contribution of Set9 to the regulation of p21 expression was also directly analyzed. The levels of p21 expression in U2OS cells stably expressing ectopic Set9 were approximately two-fold higher than in the parental strain. Consistent with p21 expression in response to Adr, a similar pattern of p21 expression was observed upon treatment of cells with 5FU (a compound that also induces DNA damage); in the presence of Set9 overexpression the levels of p21 increased over two-fold compared to the parental cells. Overexpression of a catalytically inactive form of Set9 completely ablated the induction of p21 in response to DNA damage (FIG. 3A). Moreover, siRNA-mediated reduction of the intracellular levels of Set9 also impaired p21 expression upon DNA damage in U2OS cells (FIG. 3A). These findings collectively established that Set9 is required, directly or indirectly, for the expression of p21. Unexpectedly, the introduction of either the Set9 mutant or siRNA against Set9 protein also decreased the total levels of p53, without affecting the expression of unrelated proteins, such as tubulin. Moreover, the observed stimulatory effect of Set9 on p21 gene expression extended to other p53-responsive genes such as BAX and MDM2 (FIGS. 3B and 3C, respectively).

That the intracellular levels of p53 were critically dependent on the enzymatic activity of Set9 indicated that methylation of p53-K372 might affect p53 stability. This was directly tested by pulse-chase experiments. U2OS cells were transiently transfected with wild-type or a catalytically inactive Set9 and grown in a methionine-free medium in the presence of ³⁵S-methionine for 30 minutes. The cells were then washed, resuspended in media containing methionine and then a fraction of the cells were withdrawn at the indicated times. The amount of ³⁵S-radiolabeled p53 in the cytosolic and nuclear pellet fractions was analyzed by immunoprecipitation of p53 using monoclonal antibody. The stability of nuclear p53 increased in cells expressing wild-type Set9, but not in cells expressing a catalytically inactive form of Set9. Consistent with the findings that methylated p53-Lys372 is nuclear, p53 stabilization was apparent only in the fraction of nuclear p53 associated with chromatin. Thus, it was concluded that Set9-mediated methylation of p53-Lys372 resulted in the stabilization of a chromatin-bound fraction of p53. This conclusion was supported by experiments performed in H1299 cells lacking endogenous p53. Increased stabilization of wild-type p53, but not its methylation-defective mutant (Lys372Arg) was observed in these cells upon overexpression of Set9. The expression levels of ectopic wild-type and Lys372Arg mutant p53 genes were similar, as shown by RT-PCR, and thus cannot account for the observed difference in the p53 protein levels. Moreover, Set9 overexpression resulted in increased expression of the p21 gene specifically in the presence of wild-type p53, but not with the Lys372Arg mutant, confirming that Set9 regulates p53-dependent genes through p53 methylation at Lys372.

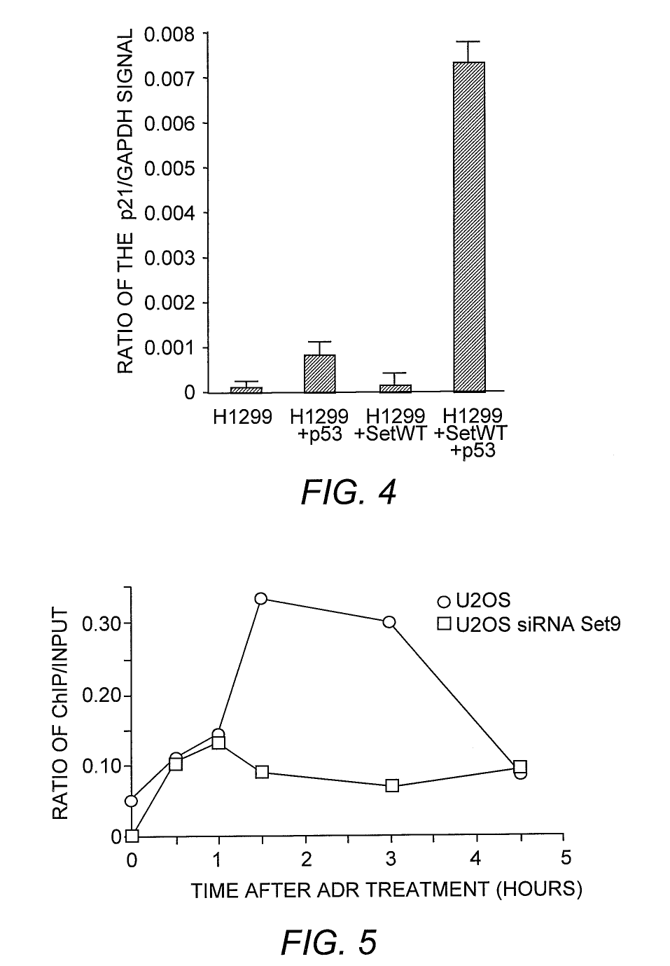

Given that overexpression of wild-type Set9 resulted in ‘hyper-stabilization’ and activation of nuclear p53, it was contemplated that induction of cell-cycle arrest and apoptosis would ensue. This was directly tested by measuring the p53-dependent apoptotic response induced by DNA damage. The results of this analysis indicated that treatment of U2OS cells with Adr increased the number of cells positively stained for Annexin V. Moreover, cells overexpressing Set9 exhibited higher apoptotic staining even without DNA damage. The treatment of Set9-overexpressing cells with Adr further increased the Annexin V staining. Overexpression of the catalytically inactive Set9 abrogated DNA-damage-induced apoptosis, indicating that the methyltransferase activity of Set9 is critical for induction of p53-dependent apoptosis. Again, the overexpression of a catalytically inactive Set9 mutant protein resulted in decreased p53 levels, confirming a functional interaction between p53 and Set9. Furthermore, Set9-mediated regulation of cell cycle and apoptosis was p53-dependent, because in the absence of p53, transfected Set9 was unable to induce p21 gene expression in H1299 (FIG. 4) or apoptosis in Saos-2 cells in response to DNA damage.

It was subsequently demonstrated that p53 methylated by Set9 is present at the promoters of p53 target genes. It was observed that p53 methylation likely precedes acetylation. Therefore, it was expected that p53 methylation must occur at very early times after DNA damage. To demonstrate this, normal U2OS and Set9-siRNA-expressing cells were treated with Adr at different times (FIG. 5). At the indicated time periods, cells were harvested, and DNA-protein complexes were cross-linked and subjected to chromatin immunoprecipitation (ChIP) analysis, initially using anti-p53-Lys372me antibodies. To increase the specificity of ChIP, the anti-p53-Lys372me immunoprecipitates were eluted from the beads with 1% SDS, diluted with immunoprecipitation buffer and then subjected to a second round of immunoprecipitation using anti-p53 antibody. The results of this analysis indicated that the amount of methylated p53 bound to the p21 promoter increased as early as 1.5 hours after DNA damage. Furthermore, Lys372 methylation was Set9-specific, because U2OS cells expressing siRNA against Set9 did not exhibit a significant increase in methylated p53 at the p21 promoter. These results indicated that methylation of p53, in addition to acetylation and phosphorylation, represents an important DNA-damage-induced modification mark required for p53 function in vivo.

It has now been demonstrated that Set9 methylates p53 at a specific lysine residue in vivo. Furthermore, this methylation is required for p53 stabilization. Not wishing to be bound by theory, it is believed that methylation interferes with MDM2-mediated ubiquitination of the six lysine residues at the C-terminus, which leads to subsequent p53 degradation and/or nuclear export (Appella & Anderson (2001) supra). Yet, because of its small size, the mono-methylation mark itself is unlikely to affect ubiquitination. Similar to histones, it is believed that certain factors may bind methylated p53 and interfere with Mdm2-dependent ubiquitination. Overexpression of the catalytically inactive Set9 mutant decreased intracellular levels of p53 and attenuated its activity. This confirms the functional connection between Set9 and p53 and also highlights another possible mechanism for p53 inactivation in human cancers. Moreover, based on the observation that p53 is a better substrate for Set9 than histone H3, it is contemplated that Set9 regulates the function of other factors. Likewise, the activities of other known lysine methyltransferases may not be limited to histones, but may also target factors important in many cellular processes. In this regard, the ternary complex of Set9 with p53 reveals the molecular basis for Set9 substrate recognition, which can be used in the identification of new Set9 targets.

Having demonstrated a novel mechanism of p53 regulation through lysine methylation by Set9 methyltransferase, the present invention relates to methods for modulating p53 activity by modulating the methylation state of p53. Molecular and cellular p53 activities which can be modulated include the transcriptional activation of other genes such as p21 (also known as cyclin-dependent kinase inhibitor 1A or CDKN1A), BAX and MDM2 (also known as Mouse Double Minute) as well as the induction of apoptosis. Modulation of p53 activity is useful in the treatment of a variety of conditions, including cancers, with wild-type or defective p53 activity.

p53 has a dual role in cancer therapy. On one hand, p53 acts as a tumor suppressor by mediating apoptosis and growth arrest in response to a variety of stresses and controlling cellular senescence. On the other hand, p53 is responsible for damage to normal tissues during cancer therapies. Because many human tumors lack functional p53, the present invention not only embraces increasing the p53 activity, but also decreasing or suppressing p53 activity to protect normal p53-containing tissues.

A p53 of use in accordance with this invention can be from any source including, for example, human (see GENBANK Accession No. NP_{—}000537), dog (see GENBANK Accession No. NP_{—}001003210), mouse (see GENBANK Accession No. NP_{—}035770.1) or rat (see GENBANK Accession No. NP_{—}112251.1). In particular embodiments, human p53 activity is modulated.

The adverse effects of p53 activity on an organism are not limited to cancer therapies. p53 is activated as a consequence of a variety of stresses associated with injuries (e.g., burns), naturally occurring diseases (e.g., hyperthermia associated with fever, and conditions of local hypoxia associated with a blocked blood supply, stroke, and ischemia) and cell aging (e.g., senescence of fibroblasts), as well as a cancer therapy. Temporary p53 inhibition, therefore, also can be therapeutically effective in reducing or eliminating p53-dependent neuronal death in the central nervous system, i.e., brain and spinal cord injury; the preservation of tissues and organs prior to transplantation; preparation of a host for a bone marrow transplant; and reducing or eliminating neuronal damage during seizures, for example. See, e.g., U.S. Pat. No. 6,982,277.

Activated p53 induces growth arrest, which often is irreversible, thus mediating damage of normal tissues in response to the applied stress. Such damage could be reduced if p53 activity is temporarily suppressed shortly before, during, or shortly after, a p53-activating event. These and other p53-dependent diseases and conditions, therefore, provide an additional area for the therapeutic administration of inhibitors, which temporarily block the activity of Set9 (e.g. Set9 siRNA or methyltransferase inhibitors) and therefore the stability of p53.

p53 also plays a role in cell aging, and, accordingly, aging of an organism. In particular, morphological and physiological alterations of normal tissues associated with aging may be related to p53 activity. Senescent cells that accumulate in tissues over time are known to maintain very high levels of p53-dependent transcription. p53-dependent secretion of growth inhibitors by senescent cells accumulate in aging tissue. This accumulation can affect proliferating cells and lead to a gradual decrease in overall proliferative capacity of tissues associated with age. Suppression of p53 activity, therefore, is envisioned as a method of suppressing tissue aging.

While some embodiments embrace decreasing p53 activity, other embodiments embrace increasing p53 activity. p53 activity can be increased by contacting p53 with Set9 so that Lys372 of p53 is methylated thereby stabilizing p53 and increasing its activity. Like p53, the histone H3 lysine 4 specific methyltransferase Set9, also known as SET domain containing (lysine methyltransferase) 7, can be from the same or different source from which the p53 is obtained. For example, human (see GENBANK Accession No. AAL69901), dog (see GENBANK Accession No. XP_{—}533287.2), mouse (see GENBANK Accession No. NP_{—}542983.3), rat (see GENBANK Accession No. XP_{—}001072672.1) or chicken (see GENBANK Accession No. XP_{—}420409) Set9 methyltransferase can be employed in this invention.

Whether simulating or inhibiting p53 activity, any well-known method for monitoring gene expression can be used to detect p53-dependent transcriptional activation or suppression of genes such as p21, BAX and MDM2. For example, RNA can be detected by northern blot analysis, which involves the separation of RNA and hybridization of a complementary labeled probe. Methods for northern blot analysis are well-known in the art.

RNA expression can also be detected by enzymatic cleavage of specific structures (INVADER assay, Third Wave Technologies, Madison, Wis.). See, e.g., U.S. Pat. Nos. 5,846,717; 6,090,543; 6,001,567; 5,985,557; and 5,994,069. The INVADER assay detects specific nucleic acid (e.g., RNA) sequences by using structure-specific enzymes to cleave a complex formed by the hybridization of overlapping oligonucleotide probes.

RNA (or corresponding cDNA) can be detected by hybridization to an oligonucleotide probe. A variety of hybridization assays using a variety of technologies for hybridization and detection are available. For example, in some embodiments, TaqMan assay (Applied Biosystems, Foster City, Calif.) is utilized. See, e.g., U.S. Pat. Nos. 5,962,233 and 5,538,848. The assay is performed during a PCR reaction. The TaqMan assay exploits the 5′-3′ exonuclease activity of the AMPLITAQ GOLD DNA polymerase. An oligonucleotide probe with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

Reverse-transcriptase PCR (RT-PCR) is another suitable method for detecting the expression of RNA. In RT-PCR, RNA is enzymatically converted to complementary DNA or cDNA using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, a quantitative reverse transcriptase PCR is employed using a competitive template method described, e.g., in U.S. Pat. Nos. 5,639,606; 5,643,765; and 5,876,978.

In other embodiments, transcriptional activation of p53-dependent genes is assayed by measuring the level of polypeptide expression. The level of polypeptide expressed can be measured using any suitable method, including but not limited to, immunoassays. Suitable immunoassays of use in detecting protein levels include, but are not limited to, radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), western blot analysis, precipitation reactions, agglutination assays (e.g., gel agglutination assays, hemagglutination assays, etc.), complement fixation assays, immunofluorescence assays, protein A assays, and immunoelectrophoresis assays. In other embodiments, immunohistochemistry is utilized for the detection of antibody binding.

Antibody binding to the protein of interest can be detected by detecting a label on the primary antibody. Alternatively, the primary antibody is detected by detecting binding of a secondary antibody or reagent to the primary antibody. Many methods are known in the art for detecting binding in an immunoassay and are within the scope of the present invention.

It is further contemplated that an automated detection assay is used. Methods for the automation of immunoassays include, but are not limited to, those described in U.S. Pat. Nos. 5,885,530; 4,981,785; 6,159,750; and 5,358,691. In some embodiments, the analysis and presentation of results is also automated.

As demonstrated herein, Set9 methylation of Lys372 of p53 induces cell-cycle arrest and apoptosis in the presence and absence of DNA damaging agents. Moreover, methylation of p53 can be inhibited using an siRNA which blocks Set9 expression or a catalytically inactive Set9. Accordingly, the present invention also embraces a method for modulating apoptosis in a cell by modulating the activity of Set9. In one embodiment, the cell is contacted with Set9 so that p53 is stabilized and apoptosis in the cell is induced. In another embodiment, the cell is contacted with a Set9 inhibitor (e.g., a Set9-specific siRNA or Set9 methyltransferase inhibitor) so that p53 is destabilized and apoptosis in the cell is reduced. As disclosed herein, the stability of p53 is associated with the methylation state of Lys372, wherein the methylation of Lys372 stabilizes p53 and the lack of methylation of Lys732 destabilizes p53 thereby decreasing p53 protein levels.

Set9 can be provided to a cell in the form of a protein (see, e.g., suitable Set9 proteins disclosed herein) or a nucleic acid molecule encoding Set9 protein. Set9 protein can be delivered to a cell via any suitable protein delivery method including, but not limited to, liposomes (see, e.g., Castor (2005) Curr. Drug Deliv. 2(4):329-40), peptide-based delivery (e.g., HIV Tat peptide; see Schwartz and Zhang (2000) Curr. Opin. Mol. Ther. 2(2):162-7) or nanoparticles (see, e.g., Vasir and Labhasetwar (2005) Technol. Cancer Res. Treat. 4(4):363-74). Likewise, nucleic acid molecules encoding Set9 can be provided to a cell by any standard method including those of use in protein delivery as well as direct injection of naked DNA constructs, bombardment with gold particles loaded with said constructs, and gene delivery vehicles. Of particular use are gene delivery vehicles derived from viruses, including, but not limited to, adenoviruses, retroviruses, vaccinia viruses, and adeno-associated viruses.

For example, self-propagating adenovirus (Ad) vectors have been extensively used to deliver foreign genes to a great variety of cell types in vitro and in vivo. Self-propagating viruses are those which can be produced by transfection of a single piece of DNA (the recombinant viral genome) into a single packaging cell line to produce infectious virus; self-propagating viruses do not require the use of helper virus for propagation. As with many vectors, adenoviral vectors have limitations on the amount of heterologous nucleic acid they are capable of delivering to cells. For example, the capacity of adenovirus is approximately 8-10 kb, the capacity of adeno-associated virus is approximately 4.8 kb, and the capacity of lentivirus is approximately 8.9 kb.

In an effort to address the viral replication problems associated with first generation Ad vectors, so called second generation Ad vectors have been developed. Second generation Ad vectors delete the early regions of the Ad genome (E2A, E2B, and E4). Highly modified second generation Ad vectors are less likely to generate replication-competent virus during large-scale vector preparation, and complete inhabitation of Ad genome replication should abolish late gene replication. Host immune response against late viral proteins is thus reduced (see Amalfitano, et al. (1998) J. Virol. 72:926-933). The elimination of E2A, E2B, and E4 genes from the Ad genome also provide increased cloning capacity. The deletion of two or more of these genes from the Ad genome allows for example, the delivery of full-length or cDNA genes via Ad vectors (Kumar-Singh, et al. (1996) Hum. Mol. Genet. 5:913).

Gutted, or helper-dependent, Ad vectors contain cis-acting DNA sequences that direct adenoviral replication and packaging but do not contain viral coding sequences (see Fisher, et al. (1996) Virology 217:11-22; Kochanek, et al. (1996) Proc. Nat. Acad. Sci. USA 93:5731-5736). Gutted vectors are defective viruses produced by replication in the presence of a helper virus, which provides all of the necessary viral proteins in trans. Since gutted vectors do not contain any viral genes, expression of viral proteins is not possible. Gutted Ad vectors are able to maximally accommodate up to about 37 kb of exogenous DNA, however, 28-30 kb is more typical. For example, a gutted Ad vector can accommodate a full-length gene or cDNA, but also expression cassettes or modulator proteins.

Vectors based on human or feline lentiviruses are another vector useful for gene therapy applications. Lentivirus-based vectors infect nondividing cells as part of their normal life cycles, and are produced by expression of a package-able vector construct in a cell line that expresses viral proteins. The small size of lentiviral particles constrains the amount of exogenous DNA they are able to carry to about 10 kb.

Retroviruses (family Retroviridae) are divided into three groups: the spumaviruses (e.g., human foamy virus); the lentiviruses (e.g., human immunodeficiency virus and sheep visna virus) and the oncoviruses (e.g., MLV, Rous sarcoma virus). Retroviruses are enveloped (i.e., surrounded by a host cell-derived lipid bilayer membrane) single-stranded RNA viruses that infect animal cells. When a retrovirus infects a cell, its RNA genome is converted into a double-stranded linear DNA form (i.e., it is reverse-transcribed). The DNA form of the virus is then integrated into the host cell genome as a provirus. The provirus serves as a template for the production of additional viral genomes and viral mRNAs. Mature viral particles containing two copies of genomic RNA bud from the surface of the infected cell. The viral particle comprises the genomic RNA, reverse transcriptase and other pol gene products inside the viral capsid (which contains the viral gag gene products), which is surrounded by a lipid bilayer membrane derived from the host cell containing the viral envelope glycoproteins (also referred to as membrane-associated proteins).

The organization of the genomes of numerous retroviruses is well-known in the art and this has allowed the adaptation of the retroviral genome to produce retroviral vectors. The production of a recombinant retroviral vector carrying a gene of interest is typically achieved in two stages. First, the gene of interest is inserted into a retroviral vector which contains the sequences necessary for the efficient expression of the gene of interest (including promoter and/or enhancer elements which may be provided by the viral long terminal repeats (LTRs) or by an internal promoter/enhancer and relevant splicing signals), sequences required for the efficient packaging of the viral RNA into infectious virions (e.g., the packaging signal (Psi), the tRNA primer binding site (−PBS), the 3′ regulatory sequences required for reverse transcription (+PBS) and the viral LTRs). The LTRs contain sequences required for the association of viral genomic RNA, reverse transcriptase and integrase functions, and sequences involved in directing the expression of the genomic RNA to be packaged in viral particles. For safety reasons, many recombinant retroviral vectors lack functional copies of the genes that are essential for viral replication (these essential genes are either deleted or disabled); the resulting virus is said to be replication-defective.

For the purposes of the present invention, the term gene refers to a nucleic acid (e.g., DNA) sequence that contains coding sequences necessary for the production of a polypeptide, precursor, or RNA (e.g., rRNA, tRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term gene encompasses both cDNA and genomic forms of a gene. A genomic form or clone of a gene contains the coding region interrupted with non-coding sequences termed introns or intervening regions or intervening sequences. In some embodiments, the genomic clone is directly inserted into the recombinant vector for expression in vivo. In other embodiments, a cDNA clone is employed.

Following the construction of the recombinant vector, the vector DNA is introduced into a packaging cell line. Packaging cell lines provide viral proteins required in trans for the packaging of the viral genomic RNA into viral particles having the desired host range (i.e., the viral-encoded gag, pol and env proteins). The host range is controlled, in part, by the type of envelope gene product expressed on the surface of the viral particle. Packaging cell lines may express ecotrophic, amphotropic or xenotropic envelope gene products. Alternatively, the packaging cell line may lack sequences encoding a viral envelope (env) protein. In this case the packaging cell line will package the viral genome into particles that lack a membrane-associated protein (e.g., an env protein). In order to produce viral particles containing a membrane associated protein that will permit entry of the virus into a cell, the packaging cell line containing the retroviral sequences is transfected with sequences encoding a membrane-associated protein (e.g., the G protein of vesicular stomatitis virus (VSV)). The transfected packaging cell will then produce viral particles that contain the membrane-associated protein expressed by the transfected packaging cell line; these viral particles, which contain viral genomic RNA derived from one virus encapsidated by the envelope proteins of another virus are said to be pseudotyped virus particles.

The most commonly used recombinant retroviral vectors are derived from the amphotropic Moloney murine leukemia virus (MoMLV) (Miller and Baltimore (1986) Mol. Cell. Biol. 6:2895). The MoMLV system has several advantages including the ability to infect many different cell types, established packaging cell lines are available for the production of recombinant MoMLV viral particles, and the transferred genes are permanently integrated into the target cell chromosome. The established MoMLV vector systems contain a DNA vector containing a small portion of the retroviral sequence (the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the viral proteins required for particle assembly (Markowitz, et al. (1998) J. Virol. 62:1120).

The low titer and inefficient infection of certain cell types by MoMLV-based vectors has been overcome by the use of pseudotyped retroviral vectors that contain the G protein of VSV as the membrane associated protein. Unlike retroviral envelope proteins which bind to a specific cell surface protein receptor to gain entry into a cell, the VSV G protein interacts with a phospholipid component of the plasma membrane (Mastromarino, et al. (1977) J. Gen. Virol. 68:2359). Because entry of VSV into a cell is not dependent upon the presence of specific protein receptors, VSV has a broad host range. Pseudotyped retroviral vectors bearing the VSV G protein have an altered host range characteristic of VSV (i.e., they can infect almost all species of vertebrate, invertebrate and insect cells). Importantly, VSV G-pseudotyped retroviral vectors can be concentrated 2000-fold or more by ultracentrifugation without significant loss of infectivity (Burns. et al. (1993) Proc. Natl. Acad. Sci. USA 90:8033). The VSV G protein has also been used to pseudotype retroviral vectors based upon the human immunodeficiency virus (HIV) (Naldini, et al. (1996) Science 272:263). Thus, the VSV G protein may be used to generate a variety of pseudotyped retroviral vectors and is not limited to vectors based on MoMLV.

Set9 inhibitors of use in accordance with the present invention include those identified herein (i.e., Set9-specific siRNA of SEQ ID NO:5), as well as inhibitors readily identified in screening assays for agents which inhibit the expression or activity of Set9 or interaction of Set9 with p53 (e.g., an antibody which binds to unmethylated residue 372 of p53). Small molecule inhibitors can be identified from libraries of compounds or based upon known protein methylation inhibitors such as 5′-deoxy-5′-methylthioadenosine. Furthermore, it is contemplated that p53 peptide substrates such as those set forth herein as SEQ ID NO:1 and SEQ ID NO:12 can be used directly or as lead compounds in the generation peptide inhibitors which block the activity of Set9. Moreover, inhibitors can be designed based upon the crystal structure disclosed herein.

After contacting the cell with Set9 or Set9 inhibitor, the presence, absence and/or extent of apoptosis induction can be determined using any suitable method. For example, apoptosis can be detected by direct inspection for pyknotic bodies under light microscope or electron microscope. Other assays include the TUNEL assay, in which broken DNA ends are labeled preferentially; the caspase assay, in which caspase cleavage of a marker protein allows detection; the Annexin V assay, in which cell surface exposure of phosphatidylserine is measured with fluorescently (e.g., FITC or phycoerythrin) linked, phosphatidylserine binding protein, Annexin V; DNA laddering, in which certain characteristic bands are visualized in agarose gel electrophoresis; and trypan blue exclusion assay, in which a blue dye enters apoptotic cells, but is excluded from viable cells.

In some embodiments, apoptosis is modulated in a cell which expresses wild-type p53, wherein the term wild-type refers to a gene or gene product isolated from a naturally occurring source. A wild-type gene is that which is most frequently observed in a population and is thus arbitrarily designed the “normal” or “wild-type” form of the gene. In alternative embodiments, apoptosis is modulated, in particular increased, in a cell which expresses a defective p53. For the purposes of the present invention, a defective p53 is intended to include any mutation which decreases either the expression or activity of p53. In this regard, it is known that missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg (1991) Science 254:1138-1146). However, it has been shown that transfection of DNA encoding wild-type p53 into human breast cancer cell lines restores growth suppression control in such cells (Casey, et al. (1991) Oncogene 6:1791-797). A similar effect has also been demonstrated on transfection of wild-type p53 into human lung cancer cell lines (Cajot, et al. (1992) Cancer Res. 52(24):6956-60.). Exogenous p53 appears dominant over the mutant gene and will select against proliferation when transfected into cells with the mutant gene.

Therefore, in embodiments pertaining to an increase in apoptosis in a cell which expresses a defective p53, the present invention provides for contacting the cell with exogenous wild-type p53. As with Set9, wild-type p53 can be provided to a cell in the form of a protein (see, e.g., suitable p53 proteins disclosed herein) or a nucleic acid molecule encoding p53 protein. Exemplary adenovirus-mediated wild-type p53 gene transfer vectors for in vitro and in vivo applications are well-known in the art. See, e.g., Gabrilovich (2006) Expert Opin. Biol. Ther. 6(8):823-32; Clayman, et al. (1999) Clin. Cancer Res. 5:1715-1722). See also, U.S. Pat. Nos. 6,998,117 and 6,805,858, incorporated herein by reference, for details pertaining to the construction of p53 expression vectors and expression of wild-type p53 in cells.

In particular embodiments, cellular apoptosis is modulated in the presence of an antineoplastic agent. Various classes of antineoplastic (e.g., anticancer) agents are contemplated including, but not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in the present invention include, but are not limited to alkaloids such microtubule inhibitors (e.g., vincristine, vinblastine, and vindesine), microtubule stabilizers (e.g., paclitaxel (TAXOL), and docetaxel), and chromatin function inhibitors, including topoisomerase inhibitors, such as epipodophyllotoxins (e.g., etoposide (VP-16), and teniposide (VM-26)), and agents that target topoisomerase I (e.g., camptothecin and isirinotecan (CPT-11)); covalent DNA-binding agents (alkylating agents), including nitrogen mustards (e.g., metchlorethamine, chlorambucil, cyclophosphamide, ifosphamide, and busulfan (MYLERAN)), nitrosoureas (e.g., carmustine, lomustine, and semustine), and other alkylating agents (e.g., dacarbazine, hydroxymethylmelamine, thiotepa, and mitomycin); noncovalent DNA-binding agents (antitumor antibiotics), including nucleic acid inhibitors (e.g., dactinomycin (actinomycin D)), anthracyclines (e.g., daunorubicin, doxorubicin (adriamycin), and idarubicin (idamycin)), anthracenediones (e.g., anthracycline analogues such as mitoxantrone), bleomycins (BLENOXANE), and plicamycin (mithramycin); antimetabolites, including antifolates (e.g., methotrexate, FOLEX, and MEXATE), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin)), pyrimidine antagonists (e.g., fluoropyrimidines such as 5-fluorouracil (ADRUCIL), 5-fluorodeoxyuridine (FdUrd)), and cytosine arabinosides (e.g., CYTOSAR (ara-C) and fludarabine); enzymes, including L-asparaginase, and hydroxyurea; hormones, including glucocorticoids, antiestrogens (e.g., tamoxifen), nonsteroidal antiandrogens (e.g., flutamide), and aromatase inhibitors (e.g., anastrozole (ARIMIDEX)); platinum compounds (e.g., cisplatin and carboplatin); monoclonal antibodies conjugated with anticancer drugs, toxins, and/or radionuclides; biological response modifiers (e.g., interferons (e.g., IFN-alpha) and interleukins (e.g., IL-2); adoptive immunotherapy; hematopoietic growth factors; agents that induce tumor cell differentiation (e.g., all-trans-retinoic acid); gene therapy techniques; antisense therapy techniques; tumor vaccines; therapies directed against tumor metastases (e.g., batimastat); angiogenesis inhibitors; proteosome inhibitors (e.g., VELCADE); inhibitors of acetylation and/or methylation (e.g., HDAC inhibitors); modulators of NFkappaB; inhibitors of cell cycle regulation (e.g., CDK inhibitors); and ionizing radiation.

Given the role of p53 in cancer etiology, the present invention also relates to a method for treating cancer by administering to a subject in need of treatment an effective amount of Set9 to stabilize p53. As disclosed herein, Set9 can be provided to the subject in the form of Set9 protein (e.g., in liposomes, nanoparticles, etc.) or nucleic acid molecules encoding Set9 (e.g., delivered by recombinant vectors). As used herein, the term subject refers to any animal (e.g., a mammal), including, but not limited to, a human, a non-human primate, a rodent, ovine, bovine, ruminant, lagomorph, porcine, caprine, equine, canine, feline, and the like, which is to be the recipient of a particular treatment. Typically, the terms subject and patient are used interchangeably herein in reference to a human subject. A subject in need of treatment refers to a subject who has been tested and found to have cancerous cells. The cancer may be diagnosed using any suitable method, including but not limited to, biopsy, x-ray, or blood test.

As with the method for inducing apoptosis, the method of treating cancer embraces embodiments for treating cancers in which the cancer cells express a wild-type p53 or a defective p53. In this regard, treatment can also include co-administration of wild-type p53 with Set9. Wild-type p53 can be provided as p53 protein or encoded by nucleic acid molecules. Advenoviral 53 gene therapy has been shown to be well-tolerated and efficacious in the treatment of numerous cancers, both at the preclinical and clinical level (see Gabrilovich (2006) supra; Clayman, et al. (1999) supra). Methods for treating cancer and the administration of effective amounts of p53 are disclosed in U.S. Pat. Nos. 6,998,117 and 6,805,858. Moreover, other embodiments embrace the administration of Set9 in conjunction with one or more conventional antineoplastic agents as disclosed herein.

Set9 alone (protein or nucleic acid molecule), or in combination with p53 and/or an antineoplastic agent, can be formulated in pharmaceutical compositions for administration to subjects. A pharmaceutical composition of the present invention can be administered in a number of ways depending upon whether local or systemic treatment is desired and upon the area to be treated. Administration can be topical (including ophthalmic and to mucous membranes such as vaginal and rectal delivery); pulmonary by inhalation or insufflation of powders or aerosols (e.g., by nebulizer) via intratracheal or intranasal routes; oral or parenteral. Parenteral administration includes intravenous, intraarterial, subcutaneous, intraperitoneal or intramuscular injection or infusion. Moreover, intracranial, e.g., intrathecal or intraventricular, administration is also contemplated.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may also be used.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration can include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, can conveniently be presented in unit dosage form and prepared according to conventional techniques well-known in the pharmaceutical industry. Such techniques include the step of bringing into association the active ingredient(s) with the pharmaceutical carrier(s) or excipient(s). In general the formulations are prepared by uniformly and intimately bringing into association the active ingredients with liquid carriers or finely divided solid carriers or both, and then, if necessary, shaping the product.

The pharmaceutical compositions of the present invention can be formulated into any of many possible dosage forms such as, but not limited to, tablets, capsules, liquid syrups, soft gels, suppositories, and enemas. Suspensions in aqueous, non-aqueous or mixed media are also provided. Aqueous suspensions can further contain substances that increase the viscosity of the suspension including, for example, sodium carboxymethylcellulose, sorbitol and/or dextran. The suspension may also contain stabilizers.

Pharmaceutical foams such as, but not limited to, emulsions, microemulsions, creams, jellies and liposomes are also provided herein. While basically similar in nature these formulations vary in the components and the consistency of the final product.

Agents that enhance uptake of nucleic acids at the cellular level may also be added to the pharmaceutical and other compositions of the present invention. For example, cationic lipids, such as LIPOFECTIN (U.S. Pat. No. 5,705,188), cationic glycerol derivatives, and polycationic molecules, such as polylysine (WO 97/30731), also enhance the cellular uptake of nucleic acids.

Other adjunct components conventionally found in pharmaceutical compositions can also be included. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the active agents of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the pharmaceutical agents of the formulation.

Dosing may be dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual agent employed, and can generally be estimated based on EC₅₀s found to be effective in in vitro and in vivo animal models. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the agents is administered in maintenance doses.

In the context of treatment, an effective amount of Set9 is an amount which provides a detectable increase in the methylation of Lys372 of p53, p53 stability, p53-dependent gene transcription, and/or cancer cell apoptosis as compared with the factors prior to commencement of treatment. An increase in any one of these factors can be determined as disclosed herein or using any other established method. In particular, the methylation state and stability of p53 can be determined using an antibody which preferentially binds methylated p53.

In this regard, the present invention also relates to an antibody which specifically recognizes methylated p53. An antibody is said to specifically recognize methylated p53 if it is able to discriminate between the unmethylated and methylated forms p53 and preferentially bind methylated p53, in particular mono-methylated p53. In certain embodiments, the antibody of the invention binds methylated Lys372 of p53.

An antibody of the present invention can be monoclonal or polyclonal. Such antibodies can be natural or partially or wholly synthetically produced. All fragments or derivatives thereof which maintain the ability to specifically bind to and recognize the methylated p53 are also contemplated. The antibody can be a member of any immunoglobulin class, including any of the classes: IgG, IgM, IgA, IgD, and IgE. Derivatives of the IgG class, however, are preferred in the present invention.

Antibody fragments can be any derivative of an antibody which is less than full-length. Preferably, the antibody fragment retains at least a significant portion of the full-length antibody's specific binding ability. Examples of antibody fragments include, but are not limited to, Fab, Fab′, F(ab′)₂, scFv, Fv, dsFv diabody, or Fd fragments. The antibody fragment can be produced by any means. For instance, the antibody fragment can be enzymatically or chemically produced by fragmentation of an intact antibody or it can be recombinantly produced from a gene encoding the partial antibody sequence. The antibody fragment can optionally be a single-chain antibody fragment. Alternatively, the fragment can be composed of multiple chains which are linked together, for instance, by disulfide linkages. A functional antibody fragment will typically be at least about 50 amino acids and more typically will be at least about 200 amino acids. As used herein, an antibody also includes bispecific and chimeric antibodies.

Naturally produced antibodies can be generated using well-known methods (see, e.g., Kohler and Milstein (1975) Nature 256:495-497; Harlow and Lane, In: Antibodies: A Laboratory Manual, Cold Spring Harbor Laboratory, New York (1988)). Alternatively, antibodies which specifically recognize methylated p53 are derived by a phage display method. Methods of producing phage display antibodies are well-known in the art (e.g., Huse, et al. (1989) Science 246(4935):1275-81). Selection of methylated p53-specific antibodies is based on binding affinity to p53 which is either methylated, in particular mono-methylated, and may be determined by the various well-known immunoassays as disclosed herein. As indicated, antibodies of the present invention find application in the detection of the methylation state of p53 and may also be useful in the diagnosis of diseases or conditions associated with increased levels of p53.

The invention is described in greater detail by the following non-limiting examples.

### EXAMPLE 1

### Constructs

GST-H3 N-terminal peptide was expressed according to established methods (Tachibana, et al. (2001) J. Biol. Chem. 276:25309-25317). For GST-C-terminal p53, construct C-terminal (residues 290-393) was cloned into PGEX3 vector (PROMEGA, Madison, Wis.). For methylation site identification, different p53 peptides (residues 290-235, 340-364, 364-393) were cloned into Pet102 (INVITROGEN, Carlsbad, Calif.) vector and expressed in Escherichia coli as thioredoxin fusion proteins.

### EXAMPLE 2

### Peptides

The following peptides were chemically synthesized for antibody production and dot blots.

p53 unmodified: NH₂-Cys-Ser-His-Leu-Lys-Ser-Lys-Lys-Gly-Gln-Ser-Thr-COOH (SEQ ID NO:1);

p53 mono-methyl-Lys372: NH₂-Cys-Ser-His-Leu-Lys-Ser-Lys(Me)-Lys-Gly-Gln-Ser-Thr-COOH (SEQ ID NO:2);

p53 di-methyl-Lys372: NH₂-Cys-Ser-His-Leu-Lys-Ser-Lys(Me2)-Lys-Gly-Gln-Ser-Thr-COOH (SEQ ID NO:3); and

p53 tri-methyl-Lys372: NH₂-Cys-Ser-His-Leu-Lys-Ser-Lys(Me3)-Lys-Gly-Gln-Ser-Thr-COOH (SEQ ID NO:4).

### EXAMPLE 3

### Methylation and Stability Assays

Methylation assay was performed according to known methods (Nishioka, et al. (2002) Genes Dev. 16:479-489).

For stability assays, U2OS cells were transfected with wild-type or mutant Set9 36 hours before labeling. Twenty-four hours after transfection, cells were split into 60-mm plates to 50% confluence. Subsequently, cells were washed twice with phosphate-buffered saline (PBS) and preincubated for 30 minutes with DMEM (without methionine) containing 5% dialyzed fetal calf serum (FCS). Cells were labeled for 30 minutes in fresh methionine-free DMEM containing 0.5 mCi of ³⁵S methionine. Radioactive media were then removed and cells were washed with PBS, followed by the addition of DMEM containing FCS, and 2 mM methionine. At the indicated times, cells were washed with PBS and collected. Cytoplasmic and nuclear fractions were prepared using established methods (Nishioka, et al. (2002) supra) and p53 was immunoprecipitated from each fraction with p53-specific antibody (DO-1).

### EXAMPLE 4

### siRNA Construct and Set9 Knock-Down

For lentiviral vector-based knock-down, the desired 23 base pair (bp) stem-loop RNAs were expressed from the pLSL-GFP vector driven by the human H1 gene promoter (Budanov, et al. (2004) Science 304:596-600). The vector also contained a minimal histone H4 promoter that drives transcription of a green fluorescent protein (GFP) gene used to monitor infection efficiency. pLSL-GFP vectors expressing the following hairpin RNAs were co-transfected with packaging plasmids pCMV-VSVG and pCMV-deltaR8.2 into 293T cells on a 6-cm dish by standard calcium-phosphate precipitation. Viral supernatants were collected from the transfected 293T cells 24, 36 and 48 hours post-transfection, filtered and used for infection of ˜5×10⁴ target U2OS cells grown on six-well plates in the presence of 4 μg/ml of POLYBRENE (SIGMA, St. Louis, Mo.). The U2OS cells were infected with >100% efficiency as judged by GFP fluorescence, expanded and used as a mass culture for all subsequent experiments.

The stem-loop siRNAs were synthesized as two complementary 67-base oligonucleotides, annealed and cloned into BamHI/EcoRI sites of pLSL-GFP vector. The siRNA oligo used to target SET9 was 5′-gat ccg cac ctg gac gat gac gga tta cct tcc tgt caG TAA TCC GTC ATC GTC CAG GTG Ctt ttt g-3′ (SEQ ID NO:5) (targeted sequences are in upper case, the loop sequence between sense and antisense 23-mers is in lower case, restriction sites that overhang nucleotides are underlined).

### EXAMPLE 5

### RT-PCR

Total RNA was extracted using the TRI reagent (INVITROGEN) according to the manufacturer's instructions. First-strand complementary DNA was synthesized using READY-TO-GO You-Prime First-Strand Beads (Amersham Biosciences, Piscataway, N.J.).

### EXAMPLE 6

### Real-Time PCR

Real-time PCR was performed with the DNA ENGINE OPTICON 2 System (MJ Research, Waltham, Mass.) according to the manufacturer's instructions. The primers for human p21 (Cip1/WAF1′) and GAPDH (GENBANK accession numbers NM_{—}000389 and NP_{—}002037, respectively) were designed using the Primer3 program. Primers for the p21 gene: 5′-CAC CGA GAC ACC ACT GGA GG-3′ (SEQ ID NO:6) and 5′-GAG AAG ATC AGC CGG CGT TT-3′ (SEQ ID NO:7), GAPDH: 5′-GGG AAG GTG AAG GTC GGA GT-3′ (SEQ ID NO:8) and 5′-TTG AGG TCA ATG AAG GGG TCA-3′ (SEQ ID NO:9) were synthesized and purified by IDT DNA Technologies (Coralville, Iowa). Primer pairs were designed to amplify the appropriate DNA fragments using the following conditions: 10-minute initial denaturation, followed by cycles of 1 minute at 94° C., 1 minute at 62° C. and 1 minute at 72° C. Fluorescence was measured at the end of the elongation phase at 79° C. Expression levels were calculated as a ratio between p21 and GAPDH signals. Correct PCR products were confirmed by agarose gel electrophoresis (2% w/v) and melting curve analysis.

### EXAMPLE 7

### ChIP Assay

ChIP assay on U2OS and U2OS cells expressing siRNA-Set9 was performed using established methods (Barlev, et al. (2001) supra). Briefly, cells were cross-linked with 1% formaldehyde, neutralized with 0.125 M glycine and harvested by scraping. The chromatin fraction was prepared by incubating the cells in lysis buffer (10 mM Tris pH 8.0, 200 mM NaCl, 10 mM EDTA, 0.5 mM EGTA, 1 mM PMSF). The insoluble fraction was sonicated and subjected to immunoprecipitation with anti-p53-Lys372me serum. Following washes, the immunoprecipitated material was eluted with 1% SDS and 10 mM DTT, diluted ten times and re-immunoprecipitated with anti-p53 serum. DNA was then isolated and subjected to real-time PCR as described herein.

### EXAMPLE 8

### Analysis of Set9 Ternary Complex with p53

The Set9 protein was prepared from a GST fusion according to known methods (Xiao, et al. (2003) supra). A highly concentrated stock solution (100 mg/ml) of Set9 (residues 108-366) was prepared in 50 mM Tris-HCl, pH 7.0, 100 mM NaCl, and diluted to 10 mg/ml with a two-fold molar excess of p53 10-mer peptide mono-methylated at Lys4 and AdoHcy. Crystals were grown at 18° C. by vapor diffusion as hanging drops prepared by mixing equal volumes of protein complex with a reservoir solution containing 0.1 M Tris-HCl, pH 7.8 and 22% PEG 3350. Crystals were first transferred into mother liquor augmented with an additional 5% PEG 400, before plunging into liquid nitrogen. Data were collected from flash-cooled crystals at 100 K on an ADSC Q4R CCD detector at SRS Daresbury. Diffraction data were integrated and scaled using DENZO and SCALEPACK (Otwinowski & Minor (1993) In: Data Collection and Processing (eds Sawyer, et al.), SERC Daresbury Laboratory, Warrington, pp. 552-562). The structure was solved by molecular replacement using a known model (lo9s.brk) with AMORE. Subsequent refinement was performed using REFMAC version 5.0 (CCP4 (1994) Acta Crystallogr. D 50:760-763) and manual model building in 0 (Jones, et al. (1991) Acta Crystallogr. A 47:110-119).

The relevant crystallographic statistics are given in Table 1.

TABLE 1

| Space Group | P2₁ |
|---|---|
| Unit cell | a = 40.4 Å, b = 103.1 Å, |
| | c = 67.2 Å, α = 90°, |
| | β = 90.04°, γ = 90° |
| Resolution range | 20.0 Å-1.75 Å |
| Rsymm (%) | 4.7 (4.64)^{§} |
| I/σ | 24.79 (2.5)^{§} |
| Completeness (%) | 94.7 |

Final Model

| | |
|---|---|
| No. of Atoms SET9, p53(369-374), SAH | 1928, 46, 26 |
| Water molecules | 717 |
| Rworking (%)* | 18.37 |
| Rfree (%)^{∇} | 22.33 |
| rms deviation bond lengths (A), angels (°) | 0.010, 1.753 |

*Rworking = Σ||Fo| − |Fc||/Σ|Fo|.
^{∇}Rfree =
Σ_{T}|Fo| − |Fc|/σ_{T}|Fo|, where T is a test data set of 5% of the total reflections randomly chosen and set aside before refinement.
^{§}The average value across the resolution range while that in parenthesis is the value for the highest resolution bin (1.75-1.81 Å).

### EXAMPLE 9

### Determination of p53 Peptide Affinities

The dissociation constants were determined using fluorometric competition assays. The p53 and H3 peptides were unlabelled and used to displace a dansyl-labeled H3 10-mer (Ala-Arg-Thr-Lys-Gln-Thr-Ala-Arg-Lys-Tyr; SEQ ID NO:10) of known affinity. The resulting displacement curves were used to calculate the K_{d} of the unlabelled peptides: H3 20-mer (Ala-Arg-Thr-Lys-Gln-Thr-Ala-Arg-Lys-Ser-Thr-Gly-Gly-Lys-Ala-Pro-Arg-Lys-Gln-Tyr; SEQ ID NO:11) and wild-type p53 20-mer (Leu-Lys-Ser-Lys-Lys-Gly-Gln-Ser-Thr-Ser-Arg-His-Lys-Lys-Leu-Met-Phe-Lys-Thr-Tyr; SEQ ID NO:12). These peptides were tested against the Set9 construct composed of residues 52-366. Fluorescence measurements were made using a Spex Fluoromax spectrophotometer with 330 nm (excitation) and 520 nm (emission) wavelengths, respectively. The titrations were performed at 20° C. in a buffer containing 25 mM Tris-HCl, pH 8.0.

What is claimed is:

1. A method for modulating p53 activity comprising contacting p53 with Set9 so that p53 activity is modulated.

2. A method for modulating apoptosis in a cell comprising contacting a cell with an effective amount of Set9 or a Set9 inhibitor so that the p53 is respectively stabilized or destabilized and apoptosis in the cell is modulated.

3. The method of claim 2, wherein the cell expresses wild-type p53.

4. The method of claim 2, wherein the cell expresses a defective p53.

5. The method of claim 4, further comprising contacting the cell with exogenous wild-type p53.

6. The method of claim 2, further comprising contacting the cell with an antineoplastic agent.

7. A method for treating cancer comprising administering to a subject in need of treatment an effective amount of Set9 to stabilize p53 thereby treating the cancer.

8. The method of claim 7, wherein the cells of the cancer express wild-type p53.

9. The method of claim 7, wherein the cells of the cancer express a defective p53.

10. The method of claim 9, further comprising administering to the subject exogenous wild-type p53.

11. The method of claim 7, further comprising administering to the subject an antineoplastic agent.

12. An antibody which specifically recognizes methylated p53.
